(19) United States
(12) Patent Application Publication
(10) Pub. No.: US 2019/0127314 A1
(43) Pub. Date: May 2, 2019

(54) HOMOVANILLIC ACID ESTER FOR REDUCING OR INHIBITING FATTY ACID ABSORPTION IN THE SMALL INTESTINE

(71) Applicant: SYMRISE AG, Holzminden (DE)

(72) Inventors: Joachim HANS, Holzminden (DE); Barbara LIEDER, Wien (AT); Katrin GEISSLER, Einbeck (DE); Fabia HENTSCHEL, Holzminden (DE); Jakob Peter LEY, Holzminden (DE); Veronika SOMOZA, Weidling (AT)

(21) Appl. No.: 16/086,686

(22) PCT Filed: Mar. 23, 2016
(86) PCT No.: PCT/EP2016/056421

§ 371 Date: Sep. 25, 2018

(51) Int. Cl. C07C 69/734; A23L 33/00; A23L 33/20; A23L 33/10; A23L 2/52; A23L 2/02; A23G 4/06; A23G 3/36; A23C 9/13; A23C 9/18; A23C 9/152

(52) U.S. Cl. CPC C07C 69/734; A23L 33/30; A23L 33/20; A23L 33/10; A23L 2/52; A23V 2002/00; A23G 4/06; A23G 3/36; A23C 9/1307; A23C 9/18; A23C 9/1522; A23L 2/02

(57) ABSTRACT

The present invention particularly relates to new uses of compounds of formula (I) (as described herein) for inhibiting or reducing the absorption of free fatty acids in the small intestine, as well as a new compound of formula (I) as such, aroma compositions and new compositions comprising such a compound of formula (I). Further aspects of the present invention result from the patent claims and the subsequent description including the examples.

The present invention particularly relates to new uses and applications of compounds of formula (I) (as described herein), among others a new compound of formula (I) as such, aroma compositions and new compositions comprising such a compound of formula (I). Further aspects of the present invention result from the patent claims and the following descriptions including the examples.

Excess weight in humans, especially in industrial nations, is a steadily increasing medical and social problem. Due to a lack of movement and excess food intake, the incidence of chronic diseases such as obesity, insulin resistance, disorders of fat metabolism and high blood pressure strongly increases, whereby secondary diseases such as type II diabetes, myocardial or cerebral infarctions lead to an increased mortality rate.

The currently widely distributed method of calorie reduction (supplementation of high caloric carbohydrates e.g. by sweeteners, supplementation of high caloric fats e.g. by thickeners) often lead to an increased food intake and—related to that—an increased calorie uptake, whereby the intended effect of a calorie reduction is revoked. Therefore, solutions, by which the intake of calorically relevant food components, particularly fats, can be reduced, are to be preferred. This results in a stronger activation and use of the endogenous adipose reserves and additionally, the total number of adipocytes is at least not further increasing.

Desired are solutions enabling to positively influence the stabilization or, respectively, reduction of the body weight during normal nutrition. The modulation of fatty acid resorption in the human intestine, particularly in the small intestine, is a possible target for achieving such a positive influence on the stabilization or, respectively, reduction of the body weight in humans. Particularly desired are solutions including the use of substances already approved as flavourings and classified as toxicologically harmless, to broadly exclude possible side-effects (as e.g. when using pharmaceutically active substances).

The human cell line Caco-2 (human intestinal cell line) has proven useful as model system for investigating and testing intestinal resorption processes in numerous studies. Thus, in Riedel et al. (J Nutr Biochem 25 (2014) 750-757) nicotinic acid derivatives were analysed with regard to their ability of modulating fatty acid resorption by using a Caco-2-cell model. The test system could be validated by the measurements, however the inhibition of fatty acid resorption by the used substances was at maximum 15%.

EP 2767174 A1 describes the identification of alkamides, which can reduce the resorption of fatty acids in adipocytes by 5-10% in a range of 0.01-10 μM; an inhibition of the resorption in the intestinal area is however not disclosed therein. Further, alkamides are characterized by their strong sensory profiles (spiciness, tingling, etc.), which complicate a use in many foodstuffs or do not allow them at all.

US 2010/0305106 A1 discloses inhibitors of fatty acid resorption of the class of benzyliden-3-phenyl-2-thioxo-thiazolidinones or, respectively, -diones. However, these substances are structured as therapeutics in existing diseases and are also not compatible for the use in foodstuffs.

Li et al. (J Lip Res 49 (2008) 230-244) describe fatty acid inhibitors of the class of tricyclic phenothiazines, which are however not suitable for use in foodstuffs due to their psychotropic effects and their use in the therapy of schizophrenia.

Thus, there is a need for substances effecting a good inhibition of the intestinal fatty acid resorption, preferably with a low sensory potential and which are preferably compatible with the use in foodstuffs.

In the scope of own investigations, it was found that a compound of formula (I)

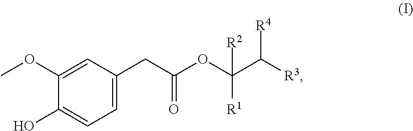

wherein
(i) R¹ and R² denote independent of each other a hydrogen atom or an alkyl group with 1-2 carbon atoms,
   R³ and R⁴ denote independent of each other a hydrogen atom or a linear or branched alkyl group with 1 to 5 carbon atoms (for example selected from the group consisting of Methyl, Ethyl, 1-Propyl, 2-Propyl-, 1-Butyl, 2-Butyl, tert-Butyl, 2-Methylprop-1-yl, 1-, 2- or 3-Pentyl-, 2-Methylbut-1-yl, 2-Methylbut-2-yl, 3-Methylbut-1-yl and 3-Methylbut-2-yl, preferably of Methyl, Ethyl, 1-Propyl, 2-Propyl, 1-Butyl, 2-Butyl, tert-Butyl, 2-Methylprop-1-yl and 1-Pentyl), a phenyl group, an alkyl phenyl group or a phenyl alkyl group or a linear or branched alkenyl group with 2 to 4 carbon atoms (for example selected from the group consisting of Ethenyl, Prop-2-ene-1-yl, Prop-1-ene-1-yl, Prop-1-ene-2-yl, 1- or 2-Cyclopropenyl-, But-1-ene-1-yl, But-1-ene-2-yl, But-1-ene-3-yl, But-2-ene-1-yl, But-3-ene-1-yl, But-2-ene-2-yl, 2-Methylprop-1-ene-1-yl, 2-Methylprop-2-ene-1-yl, 1,3-Butadiene-1-yl, 1,3-Butadiene-2-yl, and the respective possibly applicable Z- and E-isomers) or an alkenyl phenyl group or a phenyl alkylene group,
   or
(ii) R¹ and R³ form, together with the carbon atoms connecting these, a cyclohexyl ring (the rests R² and R⁴ are then substituents of the cycloalkylring; s. e.g. formula (Ia) further below), which is optionally substituted with an additional rest R⁵, wherein R⁵ is an alkyl group with 1-2 carbon atoms,
   R² denotes a hydrogen atom or an alkyl group with 1-2 carbon atoms,
   R⁴ denotes a hydrogen atom or a linear or branched alkyl group with 1 to 5 carbon atoms (for example selected from the group consisting of Methyl, Ethyl, 1-Propyl, 2-Propyl-, 1-Butyl, 2-Butyl, tert-Butyl, 2-Methylprop-1-yl, 1-, 2- or 3-Pentyl-, 2-Methylbut-1-yl, 2-Methylbut-2-yl, 3-Methylbut-1-yl and 3-Methylbut-2-yl, preferably of Methyl, Ethyl, 1-Propyl, 2-Propyl, 1-Butyl, 2-Butyl, tert-Butyl, 2-Methylprop-1-yl and 1-Pentyl), a phenyl group, an alkyl phenyl group or a phenyl alkyl group or a linear or branched alkenyl group with 2 to 4 carbon atoms (for example selected from the group consisting of Ethenyl, Prop-2-ene-1-yl, Prop-1-ene-1-yl, Prop-1-ene-2-yl, 1- or 2-Cyclopropenyl-, But-1-ene-1-yl, But-1-ene-2-yl, But-1-ene-3-yl, But-2-ene-1-yl, But-3-ene-1-yl, But-2-ene-2-yl, 2-Methylprop-1-ene-1-yl, 2-Methylprop-2-ene-1-yl, 1,3-Butadiene-1-yl, 1,3-Butadiene-2-yl and the respective possibly applicable Z- and E-isomers) or an alkenyl phenyl group or a phenyl alkylene group,
or a physiologically acceptable salt thereof, wherein the phenolic hydroxyl group in formula (I) is deprotonated, or a mixture comprising one or more compounds of formula (I) and/or physiologically acceptable salts (particularly sodium, potassium, ammonium, calcium, magnesium or zinc salts) thereof, wherein in each, the phenolic hydroxyl group in formula (I) is deprotonated, or consisting of several different compounds of formula (I) and/or salts thereof, wherein in each, the phenolic hydroxyl group in formula (I) is deprotonated,
   is surprisingly well suited for inhibiting or reducing the absorption (resorption) of free fatty acids in the small intestine, both, with regard to therapeutic and non-therapeutic applications.

I.e. the present invention relates to non-therapeutic uses (e.g. with regard to non-therapeutic/cosmetic diets) of the previously listed substances or, respectively, substance mixtures as well as to applications thereof in therapeutic methods, preferably in a therapeutic method for preventing or treating a disease which is characterized by excessive fatty acid resorption in the small intestine. Such a disease is for example selected from the group consisting of obesity, insulin resistance, disorders of fat metabolism and high blood pressure but also possible secondary diseases such as type II diabetes, myocardial or cerebral infarctions.

The ability of modulating the fatty acid resorption/absorption (as described in the scope of the present invention) of a substance or substance mixture can be determined or, respectively, evaluated e.g. according to the method described herein, by using Caco-2-cells (s. also the example section of the present text). Preferably, in the scope of the present text it is to be assumed that substances reducing or, respectively, inhibiting the fatty acid resorption (preferably when performing the method described herein, s. example section) are also suitable for the uses or, respectively, applications according to the invention and described herein. Preferably, the human colon cell line Caco-2 (ATCC-number HTB-37) is used, after differentiation to a phenotype related to entereocytes, as cell model for the resorption of free fatty acids into the small intestine. (Caco-2-cells are preferably cultivated at 37° C. and 5% CO₂ content with Dulbecco's Modified Eagles Medium (DMEM) with 10% FBS, 2% L-Glutamine and 1% Penicillin/Streptomycin. The differentiation to enterocytes is preferably performed by an 18-days cultivation of the cells after reaching confluence.)

In the scope of the present invention, such substances and substance mixtures are preferred, which reduce the fatty acid resorption by 1% or more, preferably 5% or more.

The free fatty acids, the resorption in the small intestine of which shall be reduced or, respectively, inhibited, are preferably selected from the group of the saturated fatty acids with a chain length of more than 4 carbon atoms, preferably selected from the group consisting of butanoic acid, pentanoic acid, hexanoic acid, heptanoic acid, octanoid acid, nonanoic acid, decanoic acid, undecanoic acid, dodecanoic acid, tricecanoic acid, tetracecanoic acid, pentadecanoic acid, hexadecanoic acid, heptadecanoic acid, octadecanoid acid, nonadecanoic acid, eicosanoic acid, heneicosanoic acid, docosanoic acid, tetracosanoic acid, hexacosanoic acid, octacosanoic acid, triacontanoic acid, dotriacontanoic acid and tetratriacontanoic acid. In case of a mixture of different compounds of formula (I) (as described herein), it applies in the scope of the present text that the different compounds can be e.g. not only compounds of different molecular formula, but also different stereoisomers with identical molecular formula.

It is to be noted here, that the herein described advantages and effects of the compounds of formula (I) usually also apply for their salts (as described herein) accordingly.

An application, as described above, is preferred, wherein for the compound of formula (I) or, respectively, one, more or all compounds of formula (I) independent of each other and in the mixture applies:
R¹ and R² are each a hydrogen atom,
R³ denotes a hydrogen atom or a linear or branched alkyl group with 1 to 4 carbon atoms or a phenyl group, an alkyl phenyl group or a phenyl alkyl group or an alkenyl phenyl group or a phenyl alkenyl group,
R⁴ is a hydrogen atom.

Particularly preferably, the compound of formula (I) or, respectively, one, more or all compounds of formula (I) in the mixture is/are selected from the group consisting of
2-Phenylethyl-2-(4-hydroxy-3-methoxy-phenyl)acetate (1)

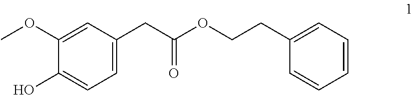 [SMILES: COc1cc(CC(=O)OCCc2ccccc2)ccc1O]

[(E)-Cinnamyl]-2-(4-hydroxy-3-methoxy-phenyl)acetate (2)

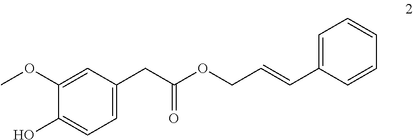 [SMILES: COc1cc(CC(=O)OC/C=C/c2ccccc2)ccc1O]

[(E)-Hex-2-enyl]-2-(4-hydroxy-3-methoxy-phenyl)acetate (5)

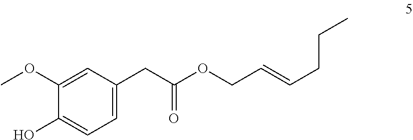 [SMILES: CCC/C=C/COC(=O)Cc1ccc(O)c(OC)c1]

[(Z)-Hex-3-enyl]-2-(4-hydroxy-3-methoxy-phenyl)acetate (6)

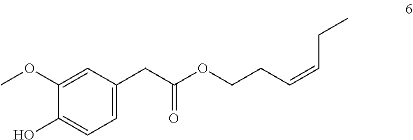 [SMILES: CC/C=C\CCOC(=O)Cc1ccc(O)c(OC)c1]

Heptyl-2-(4-hydroxy-3-methoxy-phenyl)acetate (13)

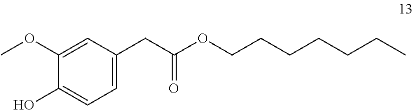 [SMILES: CCCCCCCOC(=O)Cc1ccc(O)c(OC)c1]

Ethyl-2-(4-hydroxy-3-methoxy-phenyl)acetate (16)

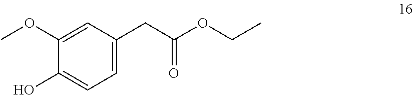

Propyl-2-(4-hydroxy-3-methoxy-phenyl)acetate (17)

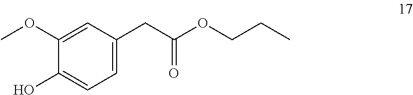 [SMILES: CCCOC(=O)Cc1ccc(O)c(OC)c1]

Butyl-2-(4-hydroxy-3-methoxy-phenyl)acetate (18)

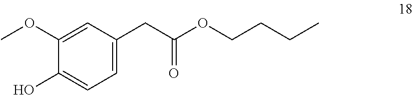 [SMILES: CCCCOC(=O)Cc1ccc(O)c(OC)c1]

Pentyl-2-(4-hydroxy-3-methoxy-phenyl)acetate (19)

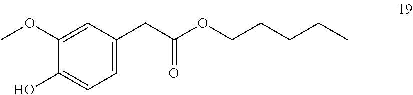 [SMILES: CCCCCOC(=O)Cc1ccc(O)c(OC)c1]

Hexyl-2-(4-hydroxy-3-methoxy-phenyl)acetate (20)

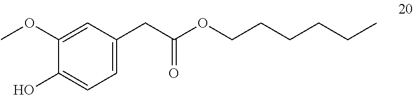 [SMILES: CCCCCCOC(=O)Cc1ccc(O)c(OC)c1]

3-Phenylpropyl-2-(4-hydroxy-3-methoxy-phenyl)acetate (21)

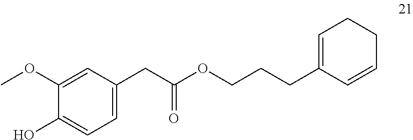 [SMILES: COc1cc(CC(=O)OCCCC2=CCCC=C2)ccc1O]

4-Phenylbutyl-2-(4-hydroxy-3-methoxy-phenyl)acetate (22)

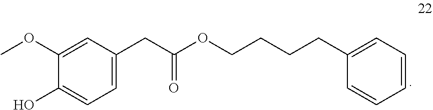 [SMILES: COc1cc(CC(=O)OCCCCc2ccccc2)ccc1O]

Also preferred is an application as described above, wherein for the compound of formula (I) or, respectively, one, more or all compounds of formula (I) independent of each other and in the mixture applies:
(i) R¹ and R² are independent of each other a hydrogen atom or Methyl or Ethyl,
   R³ and R⁴ are independent of each other a hydrogen atom or a linear or branched alkyl group with 1 to 5 carbon atoms,
   or
(ii) formula (I) corresponds to the following formula (Ia)

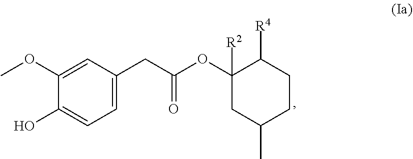

R² is a hydrogen atom,
R⁴ is 2-propyl.

Particularly preferably, the compound of formula (I) or, respectively, one, more or all compounds of formula (I) in the mixture is/are selected from the group consisting of
1-Ethylbutyl-2-(4-hydroxy-3-methoxy-phenyl)acetate (3)

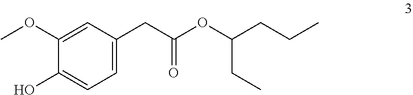 [SMILES: CCCC(CC)OC(=O)Cc1ccc(O)c(OC)c1]

3-Methylbut-2-enyl-2-(4-hydroxy-3-methoxy-phenyl)acetate (4)

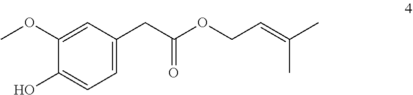 [SMILES: COc1cc(CC(=O)OCC=C(C)C)ccc1O]

Isopropyl-2-(4-hydroxy-3-methoxy-phenyl)acetate (7)

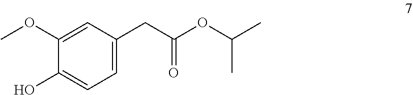 [SMILES: COc1cc(CC(=O)OC(C)C)ccc1O]

sec-Butyl-2-(4-hydroxy-3-methoxy-phenyl)acetate (8)

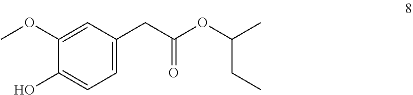 [SMILES: CCC(C)OC(=O)Cc1ccc(O)c(OC)c1]

Isobutyl-2-(4-hydroxy-3-methoxy-phenyl)acetate (9)

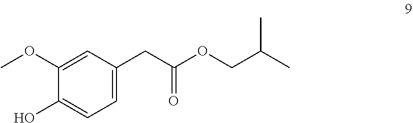 [SMILES: COc1cc(CC(=O)OCC(C)C)ccc1O]

Isopentyl-2-(4-hydroxy-3-methoxy-phenyl)acetate (10)

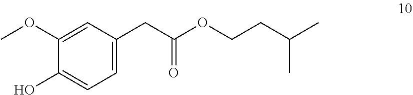 [SMILES: COc1cc(CC(=O)OCCC(C)C)ccc1O]

2-Methylbutyl-2-(4-hydroxy-3-methoxy-phenyl)acetate (11)

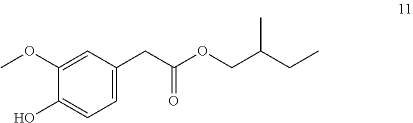 [SMILES: CCC(C)COC(=O)Cc1ccc(O)c(OC)c1]

1-Methylpentyl-2-(4-hydroxy-3-methoxy-phenyl)acetate (12)

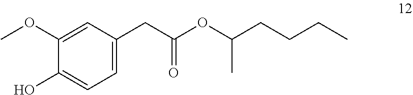 [SMILES: CCCCC(C)OC(=O)Cc1ccc(O)c(OC)c1]

1-Methyl hexyl-2-(4-hydroxy-3-methoxy-phenyl)acetate (14)

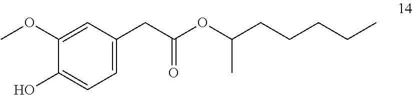 [SMILES: CCCCCC(C)OC(=O)Cc1ccc(O)c(OC)c1]

(2-Isopropyl-5-methyl-cyclohexyl)-2-(4-hydroxy-3-methoxy-phenyl)acetate (15)

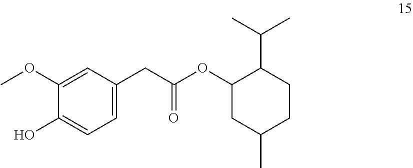 [SMILES: COc1cc(CC(=O)OC2CC(C)CCC2C(C)C)ccc1O]

2-Ethylbutyl-2-(4-hydroxy-3-methoxy-phenyl)acetate (23)

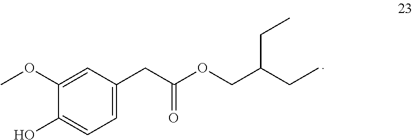 [SMILES: CCC(CC)COC(=O)Cc1ccc(O)c(OC)c1]

The compounds described herein are preferably suitable for the application (particularly as described above) in a pharmaceutical composition, a composition serving for food or pleasure, preferably wherein the total amount of compound(s) of formula (I) and/or salt(s) thereof in the composition is sufficient to inhibit or reduce the resorption of free fatty acids in the intestine. Preferably, the total amount of compound(s) of formula (I) and/or salt(s) thereof is in a range of from 1-100 ppm, further preferably 5 to 850 ppm, particularly preferably 10-20 ppm, related to the total weight of the composition.

A further aspect of the present invention relates to a new compound of formula (I) (as described above) or a salt thereof, wherein the phenolic hydroxyl group is deprotonated, or to a mixture comprising one or more different compounds of formula (I) (as defined above) and/or one or more physiologically acceptable salts thereof, wherein in each the phenolic hydroxyl group is deprotonated, or consisting of more different compounds of formula (I) (as defined above) and/or physiologically acceptable salts thereof, wherein in each the phenolic hydroxyl group is deprotonated, wherein the or, respectively, a compound of formula (I) is 2-Ethylbutyl-2-(4-hydroxy-3-methoxy-phenyl)acetate (23)

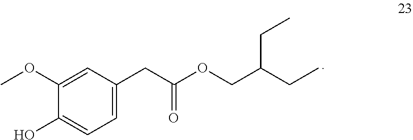 [SMILES: CCC(CC)COC(=O)Cc1ccc(O)c(OC)c1]

The present invention also relates to new aroma compositions, i.e. such comprising the new compound of formula (I) (2-Ethylbutyl-2-(4-hydroxy-3-methoxy-phenyl)acetate (23)) as named above, or a salt thereof, wherein the phenolic hydroxyl group is deprotonated, or a mixture comprising one or more different compounds of formula (I) (as defined above) and/or one or more physiologically acceptable salts thereof, wherein in each the phenolic hydroxyl group is deprotonated, or consisting of more different compounds of formula (I) (as defined above) and/or physiologically acceptable salts thereof, wherein in each the phenolic hydroxyl group is deprotonated, wherein the or, respectively, a compound of formula (I) is 2-Ethylbutyl-2-(4-hydroxy-3-methoxy-phenyl)acetate (23), preferably also comprising one or more further flavourings not according to formula (I).

The present invention further relates to a pharmaceutical composition, a composition serving for food or pleasure, comprising the new compound of formula (I) (2-Ethylbutyl-2-(4-hydroxy-3-methoxy-phenyl)acetate (23)) as named above, or a salt thereof, wherein the phenolic hydroxyl group is deprotonated, or a mixture comprising one or more different compounds of formula (I) (as defined above) and/or one or more physiologically acceptable salts thereof, wherein in each the phenolic hydroxyl group is deprotonated, or consisting of more different compounds of formula (I) (as defined above) and/or physiologically acceptable salts thereof, wherein in each the phenolic hydroxyl group is deprotonated, wherein the or, respectively, a compound of formula (I) is 2-Ethylbutyl-2-(4-hydroxy-3-methoxy-phenyl)acetate (23), or an aroma composition as described above.

Preferably, a composition according to the invention also comprises
one or more typical basic materials, excipients and additives in an amount of, related to the total weight of the composition, from 5 to 99.9999 wt.-%, preferably from 10 to 80 wt.-%,
and/or
water in an amount of, related to the total weight of the composition, up to 99.9999 wt.-%, preferably in an amount of from 5 to 80 wt.-%.

Preferred, according to the invention, is also a composition as described above, wherein the total amount of compound(s) of formula (I) and/or salt(s) thereof in the composition is sufficient to inhibit or reduce the absorption of free fatty acids in the small intestine.

Compositions serving for food or pleasure according to the invention are e.g. bakery products (e.g. bread, dry cookies, cake, other pastries), confectionery products (e.g. chocolates, chocolate bar products, other bar products, fruit gum, hard and soft caramels, chewing gum), alcoholic or non-alcoholic drinks (e.g. cocoa, coffee, green tea, black tea, (green or black) tea drinks enriched with (green or black) tea extracts, Rooibos tea, other herbal infusions, wine, drinks containing wine, beer, drinks containing beer, liqueurs, schnapps, brandies, lemonades containing fruits, isotonic drinks, refreshing drinks, nectars, fruit and vegetable juices, fruit or vegetable juice preparations), instant drinks (e.g. instant cocoa drinks, instant tea drinks, instant coffee drinks), meat products (e.g. ham, fresh sausage products or raw sausage products, spiced or marinated fresh or salt meat products), eggs or egg products (dry egg, protein, yolk), cereal products (e.g. breakfast cereals, muesli bars, precooked prepared rice products), milk products (e.g. full-fat milk drinks, milk drinks with reduced fat or without fat, rice pudding, yoghurt, kefir, cream cheese, soft cheese, hard cheese, dry milk powder, whey, butter, butter milk, partially or completely hydrolysed products containing milk protein), products made of soy protein or other soy bean fractions (e.g. soy milk and products made thereof, dinks containing isolated or enzymatically treated soy protein, drinks containing soy flour, compositions containing soya lecithin, fermented products such as tofu or tempeh or products made thereof and mixtures with fruit compositions and facultatively flavours) fruit compositions (e.g. jams, fruit ice cream, fruit sauces, fruit fillings), vegetable products (e.g. ketchup, sauces, dry vegetables, frozen vegetables, precooked vegetables, boiled down vegetables), snacks (e.g. baked or fried potato chips or potato dough products, extrudates on maize or peanut base), products on fat and oil base or emulsions thereof (e.g. mayonnaise, tartar sauce, dressings, each full-fat or fat reduced), other instant meals and soups (e.g. dry soups, instant soups, precooked soups), spices, seasoning mixes as well as particularly seasonings, which are for example used in the field of snacks, sweetener preparations, sweetener tablets or sweetener sachets, other compositions for sweetening or whitening of drinks or other foodstuffs. The compositions according to the invention can also serve as semi-finished products for producing further compositions serving for food or pleasure.

Chewing gums (as example for compositions according to the invention) generally comprise a chewing gum base, i.e. a chewable base becoming plastic while chewing, sugar of differend kinds, sugar substitutes, sweeteners, sugar alcohols, taste correctants for unpleasant taste impressions, taste correctants for usually not unpleasant taste impressions, taste modulating substances (e.g. inositol phosphate, nucleotides such as guanosin monophosphate, adenosine monophosphate or other substances such as sodium glutamate or 2-phenoxyproprionic acid), cooling substances, humectants, thickeners, emulsifiers, aromas and stabilizers or odor correctants.

Pharmaceutical compositions comprise a pharmaceutically active substance. Advantageous pharmaceutically active substances are for example steroidal anti-inflammatory substances of the corticosteroid type such as e.g. hydrocortisone, hydrocortisone derivatives such as hydrocortisone-17-butyrate, dexamethasone, dexamethasone phosphate, methylprednisolone or cortisone. Advantageous non-steroidal pharmaceutically active substances are for example inflammation inhibitors such as oxicame, such as piroxicam or tenoxicam; salicylates such as Aspririn® (acetyl salicylic acid), disalcid, solprin or fendosal, acetic acid derivatives such as diclofenac, fenclofenac, indomethacin, sulindac, tolmetin or clindanac; fenamates such as mefenamic, meclofenamic, flufenamic or niflumic; propionic acid derivatives such as ibuprofen, naproxen, flurbiprofen, benoxaprofen or pyrazoles such as phenylbutazone, oxyphenylbutazone, febrazone or azapropazone.

Particularly preferred pharmaceutical compositions are not those available only on prescription and freely marketed medical drugs, so-called OTC (over the counter) preparations, containing active substances such as paracetamol, acetyl salicylic acid or ibuprofen, vitamins (for example vitamin H, vitamins of the B-group such as vitamin B1, B2, B6, B12, niacin, pantothenic acid, preferably in form of (effervescent) tablets or capsules), minerals (preferably in form of (effervescent) tablets or capsules) such as iron salts, zinc salts, selenium salts, products containing active substances or extracts of ribwort (e.g. in cough syrup) or St. John's wort.

The compositions can also be present in form of capsules, tablets (non-coated as well as coated tablets, e.g. enteric coatings), lozenges, granules, pellets, solid substance mixtures, dispersions in liquid phases, as emulsions, as powder, as solutions, as pastes or as other swallowable or chewable compositions as well as a composition with functional ingredients as food supplement or as balanced diet.

Examples for typical basic materials, excipients and additives for compositions according to the invention are water, mixtures of fresh or processed, vegetable or animal basic or raw materials (e.g. raw, roasted, dried, fermented, smoked and/or cooked meat, bone, cartilage, fish, vegetable, fruits, herbs, nuts, vegetable or fruit juices or pastes or their mixtures), digestible or non-digestible carbohydrates (e.g. sucrose, maltose, fructose, glucose, dextrins, amylose, amylopectin, inulin, xylanes, cellulose), sugar alcohols (e.g. sorbit), natural or hardened fats (e.g. sebum, lard, palm fat, coconut fat, hardened vegetable fat), oils (e.g. sunflower oil, peanut oil, corn oil, olive oil, fish oil, soy oil, sesame oil), fatty acids or their salts (potassium stearate), proteinogenic or non-proteinogenic amino acids and related compounds (e.g. taurine), peptides, native or processed proteins (e.g. gelatine), enzymes (e.g. peptidases), nucleic acids, nucleotides, taste correctants for unpleasant taste impressions (e.g. hesperetin, Phloretin or other hydroxychalcon derivatives to be used according to US 2008/0227867as well as optionally the lactones described therein), taste correctants for typically not unpleasant taste impressions, taste modulating substances (e.g. inositol phosphate, nucleotides such as guanosin monophosphate, adenosine monophosphate or other substances such as sodium glutamate or 2-phenoxypropionic acid), emulsifiers (e.g. lecithins, diacylglycerols), stabilizers (e.g. carrageenan, alginate), preservatives (e.g. benzoic acid, sorbic acid) anti-oxidants (e.g. tocopherol, ascorbic acid), chelators (e.g. citric acid), organic or inorganic acidifiers (e.g. malic acid, acetic acid, citric acid, tartaric aid, phosphoric acid, lactic acid), additional bitter substances (e.g. chinine, caffeine, limonine, amarogentine, humolone, lupolone, catechines, tannins), sweeteners (e.g. saccharine, cyclamate, aspartame, neotame, steviosides, rebaudiosides, acesulfame K, neohesperidine dihydrochalcone, thaumatine, superaspartame), mineral salts (e.g. sodium chloride, potassium chloride, magnesium chloride, sodium phosphates), substances inhibiting enzymatic tanning (e.g. sulphite, ascorbic acid), essential oils, plant extracts, natural or synthetic dyes or colour pigments (e.g. carotinoids, flavonoids, anthocyanes, chlorophyll and their derivatives), spices, synthetic, natural or nature identical flavourings or aromatic substances as well as odour correctants.

The present invention also relates to a method for producing a pharmaceutical composition, a composition serving for foodstuff or pleasure, preferably a composition according to the invention, particularly such as described as preferred herein, comprising the following steps:
i) providing 2-ethylbutyl-2-(4-hydroxy-3-methoxy-phenyl)acetate (23) or a salt thereof, wherein the phenolic hydroxyl group is deprotonated, or a mixture comprising one or more different compounds of formula (I) (as defined above) and/or one or more physiologically acceptable salt(s) thereof, wherein in each the phenolic hydroxyl group is deprotonated, or consisting of more different compounds of formula (I) (as defined above) and/or physiologically acceptable salts thereof, wherein in each the phenolic hydroxyl group is deprotonated, wherein the or, respectively, a compound of formula (I) is 2-ehtylbutyl-2-(4-hydroxy-3-methoxy-phenyl)acetate (23), or an aroma composition as described above,
ii) providing one or more further components (s. also above) of the composition to be produced, and
iii) contacting or mixing the further components provided in step ii) with the component(s) provided in step i).

The described compositions according to the invention are preferably produced by adding the ester(s) of homovanillic acid to be used according to the invention as substance as solution nor form of an aroma composition into a pharmaceutical base composition serving for food or pleasure or into an oral pharmaceutical base composition. Advantageously, compositions according to the invention and present as solution can also be converted into a solid composition, e.g. by spray drying.

According to a preferred embodiment, compounds of formula (I) used according to the invention, or, respectively, their salts or mixtures thereof or an aroma composition according to the invention and optionally other compounds according to the invention in form of emulsions are added to liposomes, e.g. based on phosphatidyl choline, in microspheres, in nanospheres or also to capsules, granules or extrudates of a matrix suitable for foodstuff and luxury food e.g. made of starch, starch derivatives, cellulose or cellulose derivatives (e.g. hydroxyproypyl cellulose), other polysaccharides (e.g. alginate), natural fats, natural waxes (e.g. beeswax, carnauba wax) or of proteins, e.g. gelatine, to produce compositions according to the invention. In a preferred production method, the compounds of formula (I) or, respectively, their salts are complexed with one or more suitable complexing agents, for example with cycloglycans, e.g. cyclofructans, cyclodextrins or cyclodextrin derivatives, preferably alpha, beta and gamma cyclodextrin before being added and are used in this complexed form.

In the following, the present invention is further described by selected, concrete examples. The examples only serve for clarification of the invention, without limiting it or, respectively, without restricting it thereto. If not stated otherwise, all indications relate to the weight.

### EXAMPLES

### Example 1: Synthesis of 2-Ethylbutyl-2-(4-hydroxy-3-methoxy-phenyl)acetate (23)

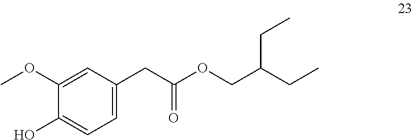 [SMILES: CCC(CC)COC(=O)Cc1ccc(O)c(OC)c1]

Homovanillic acid (2.0 g) is provided with 2-ethyl-1-butanol (equimolar) in toluene (100 mL), concentrated sulphuric acid (0.1 g) is added and heated for 5 h at the water separator until boiling. It was washed twice with saturated aqueous NaHCO₃ solution, diluted with 50 mL acetic acid, washed once with water or electively saturated aqueous NaHCO₃ solution and the solvent was removed in the vacuum. The product was obtained after column chromatographic purification on silica gel with a yield of approximately 45%.

¹H-NMR (400 MHz, CDCl₃): δ=6.85 (d, J=8.1 Hz, 1H), 6.81 (d, J=1.9 Hz, 1H), 6.76 (dd, J=8.1, 1.9 Hz, 1H), 5.56 (s, 1H), 4.01 (d, J=5.8 Hz, 2H), 3.88 (s, 3H), 3.53 (s, 2H), 1.49 (h, J=6.2 Hz, 1H), 1.38-1.25 (m, 4H), 0.86 (t, J=7.5 Hz, 6H).

¹³C-NMR (100 MHz, CDCl₃): δ=10.97 (2C), 23.26 (2C), 40.26, 41.14, 55.86, 66.86, 111.68, 114.29, 122.12, 125.98, 144.67, 146.41, 172.07.

GCMS: m/z (%)=266 [M⁺] (28), 182 (18), 137 (100), 122 (8), 107 (2), 94 (6), 85 (3), 77 (2), 66 (5), 57 (4), 43 (28).

### Example 2: Analysis of the Intestinal Fatty Acid Resorption

The resorption of free fatty acids in differentiated Caco-2-cells was analysed with the QBT Fatty Acid Uptake Kit (Molecular Devices Corporation, Germany), which uses the fluorescent fatty acid analogue BODIPY-dodecanoic acid. For preparing the assay, the differentiated Caco-2-cells are subjected to a one hour incubation in serum-free medium at standard cell culture conditions of a hunger phase. Subsequently, the test substances were added at 1 or 100 μM, diluted in Hank's balanced salt solution (HBSS of Lonza, Biozym-order number 882010)-buffer, treated with 20 mM HEPES (diluted from a 1M solution of Gibco, Thermo Fisher-order number 15630056) (in the following described as HBSS/HEPES-buffer). Controls were only treated with HBSS/HEPES-buffer. After a 30 minute preincubation at 37° C., the fluorescent fatty acid analogue was added, which was diluted in HBSS/HEPES-buffer (supplemented with 0.2% fatty acid free BSA [Sigma Aldrich, Germany]). The fluorescence signals were measured after excitation with 485 nm at an emission wavelength of 515 nm during a time of 1 h in a microtiter plate-reader (Synergy; BioTek, Germany). Starting from a time-signal-intensity curve resulting from the measurement, the area under the curve (AUC) was calculated to determine the cellular fatty acid resorption after treatment with the substances. The results obtained thereby are indicated as percent related to the control treatment (s. table 1).

| No | Name | AUC 1 μM | AUC 100 μM | Structure |
|---|---|---|---|---|
| 3 | 1-Ethylbutyl 2-(4- Hydroxy-3-Methoxy- Phenyl)acetate | 87.12 | 62.48 | 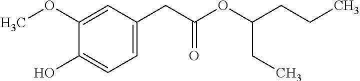 [SMILES: CCCC(CC)OC(=O)Cc1ccc(O)c(OC)c1] |
| 7 | Homovanillic acid isopropyl ester | 91.59 | 61.61 | 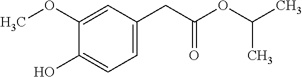 [SMILES: COc1cc(CC(=O)OC(C)C)ccc1O] |
| 8 | Homovanillic acid sec- butyl ester | 92.02 | 61.9 | 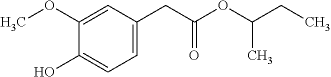 [SMILES: CCC(C)OC(=O)Cc1ccc(O)c(OC)c1] |
| 12 | 1-Methylpentyl 2-(4- Hydroxy-3-Methoxy- Phenyl)acetate | 89.55 | 53.26 | 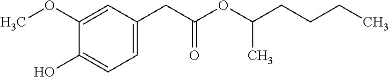 [SMILES: CCCCC(C)OC(=O)Cc1ccc(O)c(OC)c1] |
| 15 | [(1R,2S,5R)-2- Isopropyl-5-Methyl- Cyclohexyl] 2-(4- Hydroxy-3-Methoxy- Phenyl)acetate | 95.48 | 58.06 | 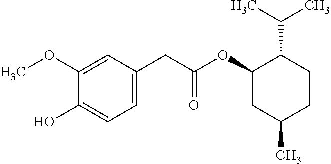 [SMILES: COc1cc(CC(=O)O[C@@H]2C[C@H](C)CC[C@H]2C(C)C)ccc1O] |
| 23 | 2-Ethylbutyl 2-(4- Hydroxy-3-Methoxy- Phenyl)acetate | 99.73 | 62.15 | 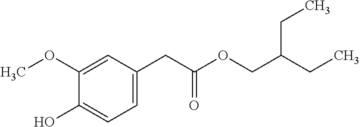 [SMILES: CCC(CC)COC(=O)Cc1ccc(O)c(OC)c1] |

Table 1: Results of the measurement of the inhibition of the fatty acid resorption in Caco-2-cells. The calculated area under the curve (AUC) is shown for tests at each 1 and 100 μM substance used (as percent of the vehicle treated control).

### Example 2: Structure-Effect-Analysis

With the test for measurement shown in example 2, in total 25 compounds of formula (I) (as described herein) were measured for their inhibitory activity regarding the fatty acid resorption in Caco-2-cells for each two concentrations (1 and 100 μM) in triple determination. The obtained activity data was subsequently used for a structure-effect-analysis by using the software-package StarDrop (Optibrium Ltd) and Vortex (Dotmatics Ltd.). Thereby, an R-group analysis with the basic structure according to formula (II) was performed. Here it was found that especially such compounds, in which R1 is branched, have a particularly good inhibition of the fatty acid resorption in the cellular test system.

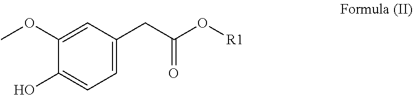

### Results:

| No | Name | AUC 1 μM | AUC 100 μM | R1 |
|---|---|---|---|---|
| 12 | 1-Methylpentyl-2-(4- Hydroxy-3-Methoxy- Phenyl)acetate | 89.55 | 53.26 | 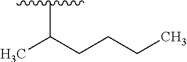 [SMILES: *C(C)CCCC] |
| 15 | [(1R,2S,5R)-2- Isopropyl-5-Methyl- Cyclohexyl]-2-(4- Hydroxy-3-Methoxy- Phenyl)acetate | 95.48 | 58.06 | 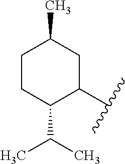 [SMILES: *C1C[C@H](C)CC[C@H]1C(C)C] |
| 7 | Homovanillic acid isopropyl ester | 91.59 | 61.61 | 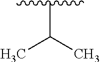 [SMILES: *C(C)C] |
| 8 | Homovanillic acid sec- butyl ester | 92.02 | 61.9 | 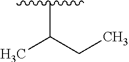 [SMILES: *C(C)CC] |
| 23 | 2-Ethylbutyl-2-(4- Hydroxy-3-methoxy- phenyl)acetate | 99.73 | 62.15 | 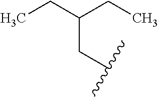 [SMILES: *CC(CC)CC] |
| 3 | 1-Ethylbutyl-2-(4- Hydroxy-3-Methoxy- Phenyl)acetate | 87.12 | 62.48 | 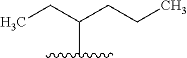 [SMILES: *C(CC)CCC] |
| 14 | 1-Methylhexyl 2-(4- Hydroxy-3-Methoxy- Phenyl)acetate | 93.38 | 67.5 | 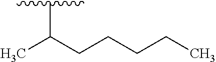 [SMILES: *C(C)CCCCC] |
| 9 | Homovanillic acid isobutyl ester | 100 | 68.01 | 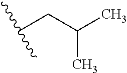 [SMILES: *CC(C)C] |
| 11 | 2-Methylbutyl-2-(4- Hydroxy-3-Methoxy- Phenyl)acetate | 98.97 | 79.02 | 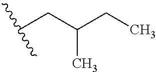 [SMILES: *CC(C)CC] |
| 4 | 3-Methylbut-2-Enyl 2- (4-Hydroxy-3-Methoxy- Phenyl)acetate | 101.2 | 80.92 | 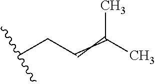 [SMILES: *CC=C(C)C] |
| 10 | Isopentyl-2-(4- Hydroxy-3-Methoxy- Phenyl)acetate | 99.85 | 82.65 | 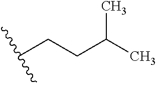 [SMILES: *CCC(C)C] |
| 17 | Homovanillic acid propyl ester | 101.2 | 89.65 | 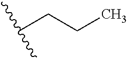 [SMILES: *CCC] |
| 6 | 3-Z-Homovanillic acid hexene ester | 99.86 | 90.36 | 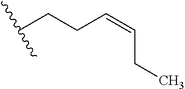 [SMILES: *CC/C=C\CC] |
| 18 | Homovanillic acid butyl ester | 102.2 | 90.69 | 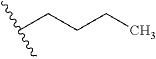 [SMILES: *CCCC] |
| 20 | Hexyl-2-(4-Hydroxy- 3-Methoxy- Phenyl)acetate | 101.9 | 91.42 | 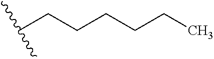 [SMILES: *CCCCCC] |
| 13 | Heptyl-2-(4-Hydroxy- 3-Methoxy- Phenyl)acetate | 103.3 | 92.68 | 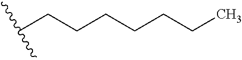 [SMILES: *CCCCCCC] |
| 19 | Homovanillic acid pentyl ester | 100.8 | 92.9 | 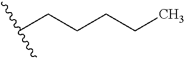 [SMILES: *CCCCC] |
| 16 | Ethyl homovanillate | 104.7 | 93.84 | 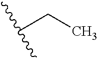 [SMILES: *CC] |
| 2 | [(E)-Cinnamyl]-2-(4- Hydroxy-3-Methoxy- Phenyl)acetate | 97.57 | 94.59 | 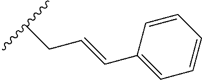 [SMILES: *C/C=C/c1ccccc1] |
| 5 | [(E)-Hex-2-Enyl]-2-(4- Hydroxy-3-Methoxy- Phenyl)acetate | 100.7 | 96.53 | 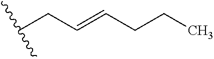 [SMILES: *C/C=C/CCC] |
| 1 | 2-Phenylethyl-2-(4- Hydroxy-3-Methoxy- Phenyl)acetate | 102.7 | 103.2 | 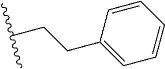 [SMILES: *CCc1ccccc1] |

### APPLICATION EXAMPLES

### Application Example 1: Refreshing Drinks (Containing Sugar, Calorie Reduced, Calorie Free)

| Ingredient | Amount in wt.-% | | | | | | |
|---|---|---|---|---|---|---|---|
| Composition | A | B | C | D | E | F | G |
| Sugar | 10 | 10 | 7 | — | — | 8 | 7 |
| (Sucrose) | | | | | | | |
| Glucose/Fructose-Syrup of | — | — | — | — | 10 | — | — |
| corn, containing 55 wt.-% | | | | | | | |
| fructose | | | | | | | |
| Rebaudioside A 95% | — | — | 0.02 | 0.05 | — | — | — |
| Citric acid | 0.15 | 0.15 | 0.06 | 0.15 | 0.15 | 0.15 | 0.15 |
| Phosphoric acid | — | — | 0.07 | — | — | — | — |
| Caramel | — | — | 0.14 | — | — | — | — |
| Caffeine | — | — | 0.01 | — | — | — | — |
| Lemon aroma | 0.1 | 0.05 | — | 0.1 | 0.1 | 0.1 | 0.1 |
| Lime aroma | — | 0.05 | — | — | — | — | — |
| Drink emulsion, “cola” type | — | — | 0.05 | — | — | — | — |
| Phloretin | — | — | 0.002 | 0.003 | — | 0.002 | 0.001 |
| Hesperetin | — | — | 0.001 | 0.002 | — | — | 0.002 |
| Extract of Rubus | — | — | — | — | — | 0.01 | — |
| suavissimus, containing 5 | | | | | | | |
| wt.-% Rubusoside related to | | | | | | | |
| the total weight of the | | | | | | | |
| extract | | | | | | | |
| Homoeriodictyol sodium salt | — | — | 0.005 | 0.005 | — | — | — |
| [(1R,2S,5R)-2-Isopropyl-5- | 0.003 | 0.001 | 0.005 | 0.01 | 0.0025 | 0.001 | 0.005 |
| Methyl-Cyclohexyl]-2-(4-Hydroxy-3-Methoxy- | | | | | | | |
| Phenyl)acetate | | | | | | | |
| Eriodictyol | — | 0.0100 | 0.0100 | — | — | 0.0100 | 0.0100 |

| | |
|---|---|
| Water | Ad 100 |

The ingredients were mixed in the listed order and water was filled up to 100%. The mixtures are filled into glass bottles and are carbonized.

### Application Example 2: Use in a Chewing Gum

| Com- | | Amount in wt.-% | |
|---|---|---|---|
| pound | Ingredient | A | B |
| A | Chewing gum base, Company | 30.4899 | 30.49999 |
| | “Jagum T” | | |
| B | Sorbit, pulverized | 39.00 | 39.00 |
| | Isomalt ® (Palatinit GmbH) | 9.50 | 9.50 |
| | Xylite | 2.00 | 2.00 |
| | Mannite | 3.00 | 3.00 |
| | Aspartam ® | 0.10 | 0.10 |
| | Acesulfam ® K | 0.10 | 0.10 |
| | Emulgum ® (Colloides | 0.30 | 0.30 |
| | Naturels, Inc.) | | |
| C | Sorbitol, 70% | 14.00 | 14.00 |
| | Glycerine | 1.00 | 1.00 |
| D | Peppermint aroma | 0.5 | 0.5 |
| | 1-Methylpentyl-2-(4-Hydroxy- | 0.01 | 0.005 |
| | 3-Methoxy-Phenyl)acetate | | |
| | Eriodictyol | 0.0100 | — |

Compounds A to D are mixed and kneaded intensively. The raw mass can be processed e.g. in form of small stripes into ready-to-eat chewing gums.

### Application Example 3: Use in Hard Caramels

| | Amount (wt.-%) | | | |
|---|---|---|---|---|
| Ingredient | A | B | C | D |
| Sugar | 74.50 | — | — | — |
| Palatinite, type M | — | 74.00 | 75.50 | 75.00 |
| Citric acid | 0.5 | 1.0 | 0.5 | — |
| Dye, yellow | — | 0.01 | — | — |
| Dye, red | — | — | 0.01 | — |
| Dye, blue | 0.01 | — | — | 0.01 |
| Peppermint aroma | 0.1 | — | — | 0.1 |
| Lemon aroma | — | 0.1 | — | — |
| Red fruit aroma | — | — | 0.1 | — |
| Rebaudioside A 98% | — | 0.040 | — | 0.040 |
| Balansine A according to | — | 0.005 | 0.010 | 0.005 |
| [SY317] | | | | |
| Hesperetin | — | 0.001 | — | 0.001 |
| Phloretin | — | 0.002 | — | — |
| 2-Ethylbutyl-2-(4-Hydroxy-3- | 0.01 | 0.0025 | 0.05 | 0.01 |
| Methoxy-Phenyl)acetate | | | | |
| Eriodictyol | 0.0100 | — | — | — |
| Water | ad 100 | ad 100 | ad 100 | ad 100 |

Palatinite or, respectively the sugar were optionally mixed with water after adding the citric acid and the mixture was molten at 165° C. and subsequently cooled down to 115° C. The aroma and the other components were added and poured into forms after mixing, removed from the forms after solidifying and subsequently packed separately.

### Application Example 4: Fat-Reduced Yoghurts

| | Composition (amount in wt.-%) | | | |
|---|---|---|---|---|
| Ingredient | A | B | C | D |
| Sucrose | 10 | 8 | 6 | — |
| Rebaudioside A 98% | — | — | — | 0.050 |
| Extract of Rubus suavissimus, | — | 0.010 | 0.010 | — |
| containing 5 wt.-% Rubusoside | | | | |
| related to the total weight | | | | |
| of the extract, e.g. of PlantExtrakt | | | | |
| Hesperetin | — | 0.001 | 0.001 | 0.002 |
| Phloretin | — | — | 0.002 | 0.002 |
| Homoeriodictyol sodium salt | — | — | — | 0.005 |
| [(1R,2S,5R)-2-Isopropyl-5-Methyl- | 0.01 | 0.01 | 0.005 | 0.01 |
| Cyclohexyl]-2-(4-Hydroxy-3-Methoxy- | | | | |
| Phenyl)acetate | | | | |
| Eriodictyol | | 0.0100 | | 0.0100 |

| | |
|---|---|
| Yoghurt, 0.1% Fat | Ad 100% |

The ingredients were mixed and cooled down to 5° C.

### Application Example 5: Fruit Gums

Composition (amount in wt.-%)

| Ingredient | A | B |
|---|---|---|
| Sucrose | 34.50 | 8.20 |
| Glucose syrup, DE 40 | 31.89 | 30.09 |
| Iso Syrup C* Tru Sweet | 1.50 | 2.10 |
| 01750 (Cerestar GmbH) | | |
| Gelatine 240 Bloom | 8.20 | 9.40 |
| Polydextrose (Litesse ® | — | 24.40 |
| Ultra, Danisco Cultor | | |
| GmbH) | | |
| Yellow and red dye | 0.01 | 0.01 |
| Citric acid | 0.20 | |
| Cherry aroma, containing 1 | — | 0.10 |
| wt.-% Hesperetin and 0.3 | | |
| wt.-% Phloretin related to | | |
| the aroma | | |
| Homovanillic acid sec | 0.01 | 0.01 |
| butyl ester | | |
| Eriodictyol | 0.0100 | |
| Water | ad 100 | ad 100 |

Polydextrose is a polysaccharide with a low calorific value, which has not a sweet taste.

### Application Example 6: Fruit Juices and Fruit Juice Drinks

| Ingredient | Amount in wt.-% | | | |
|---|---|---|---|---|
| Composition | A | B | C | D |
| Apple juice concentrate 10-fold | 10 | — | 10 | 10 |
| Recovery aroma of apple juice | 0.1 | — | 0.1 | 0.1 |
| concentrate | | | | |
| Orange juice concentrate 20-fold | — | 5 | — | — |
| Recovery aroma of orange juice | — | 0.05 | — | — |
| concentrate | | | | |
| Water | ad | ad | ad | ad |
| | 100 | 100 | 100 | 100 |
| Ascorbic acid | 0.05 | 0.05 | — | — |
| [(1R,2S,5R)-2-lsopropyl-5-Methyl- | 0.01 | 0.01 | 0.01 | 0.01 |
| Cyclohexyl] 2-(4-Hydroxy-3-Methoxy- | | | | |
| Phenyl)acetate | | | | |

### Application Example 7: Milk Mixture Drink on a Dry Base

| Ingredient | Amount in [g] | | | | | | |
|---|---|---|---|---|---|---|---|
| Composition | A | B | C | D | E | F | G |
| Sugar, fine | 5 | 5 | 5 | 5 | 5 | 5 | 5 |
| Homovanillic | 0.03 | 0.03 | 0.03 | 0.03 | 0.02 | 0.015 | 0.03 |
| acid sec butyl | | | | | | | |
| ester, 10% | | | | | | | |
| Aroma type | 0.2 | 0.2 | 0.2 | 0.2 | — | — | — |
| Cappuccino, | | | | | | | |
| spray dried | | | | | | | |
| Aroma type | — | — | — | — | 0.1 | 0.1 | 0.1 |
| strawberry, | | | | | | | |
| spray dried | | | | | | | |
| Red beet dye powder, | — | — | — | — | 0.1 | 0.08 | 0.1 |
| spray dried | | | | | | | |
| Caramel | 0.15 | 0.15 | 0.15 | 0.15 | — | — | — |
| Xanthan | 0.1 | 0.1 | 0.1 | 0.1 | 0.1 | 0.1 | 0.1 |
| Skimmed milk | 10 | 10 | 10 | — | 10 | — | — |
| powder | | | | | | | |
| Milk powder, lactose | — | — | — | — | — | 10 | 10 |
| reduced (Fa. Omira, | | | | | | | |
| Ravensburg) | | | | | | | |
| Lupinen protein | — | — | — | 5 | — | — | — |
| powder | | | | | | | |

The ingredients were mixed and packed under protective gas or in a vacuum bag. For use, the powder is stirred into 100 mL water (room temperature) and then drunk.

### Application Example 8: Spray Dried Composition as Semi-Finished Product for Aromatizing Finished Products According to the Invention

| Ingredient | Amount in wt.-% |
|---|---|
| Drinking water | 60.8% |
| Maltodextrin from wheat | 24.3% |
| Gum Arabicum | 6.1% |
| [(1 R,2S,5R)-2-Isopropyl-5-Methyl-Cyclohexyl]- | 8.8% |
| 2-(4-Hydroxy-3-Methoxy-Phenyl)acetate | |

The drinking water is provided in a container and the maltodextrin and gum arabicum are dissolved therein. Subsequently, the [(1R,2S,5R)-2-Isopropyl-5-Methyl-Cyclohexyl]-2-(4-Hydroxy-3-Methoxy-Phenyl)acetate is emulsified with a Turrax. The temperature of the spray solution should not exceed 30° C. The mixture is then spray dried (set temperature input: 185-195° C.; set temperature output: 70-75° C.).

The spray dried semi-finished product contains 18-22% [(1R,2S,5R)-2-Isopropyl-5-Methyl-Cyclohexyl]-2-(4-Hydroxy-3-Methoxy-Phenyl)acetate and can be used for producing a composition according to the invention as described herein.

### Application Example 9: Use in a Soy Drink

The compound [(1R,2S,5R)-2-Isopropyl-5-Methyl-Cyclohexyl]-2-(4-Hydroxy-3-Methoxy-Phenyl)acetate was dissolved in ethanol and added to a soy milk of a local supermarket. The mixture was then stirred in a beaker together with the milk aroma.

| Ingredient | Amount in wt.-% |
|---|---|
| Soy milk (local supermarket) | 99.8% |
| Milk aroma (Symrise) | 0.1% |
| 10% [(1R,2S,5R)-2-Isopropyl-5-Methyl-Cyclohexyl]- | 0.1% |
| 2-(4-Hydroxy-3-Methoxy-Phenyl)acetate | |

### Application Example 10: Whey Protein Drink

Production of a fruit whey protein drink according to the recipe below, with subsequent homogenisation and pasteurisation.

| Ingredient | Amount in wt.-% |
|---|---|
| Whey protein isolate | 8% |
| Fruit juice concentrate mixture (mango, | 10% |
| banana, carrot, orange) | |
| Citric acid 50% | 0.2% |
| Pectin | 0.4% |
| Vitamin mixture | 0.01% |
| Mineral salt mixture | 1% |
| 10% [(1R,2S,5R)-2-Isopropyl-5-Methyl- | 0.1% |
| Cyclohexyl]-2-(4-Hydroxy-3-Methoxy- | |
| Phenyl)acetate | |
| Mango Aroma (Symrise) | 0.05% |
| Drinking water | Ad 100% |

### Application Example 11: Drink Containing High Amounts of Protein (Sports Nutrition)

The dry ingredients are mixed and subsequently dissolved in drinking water.

| Ingredient | Amount in wt.-% |
|---|---|
| Whey protein isolate (min. 88% protein TS) | 8% |
| Amino acid mixture (45% L-Leucine; 30% L- | 1% |
| Valine, 25% L-Isoleucine) | |
| Citric acid | 0.1% |
| Sucralose | 0.007% |
| Orange dry aroma (Symrise) | 0.1% |
| 10% [(1R,2S,5R)-2-Isopropyl-5-Methyl- | 0.1% |
| Cyclohexyl]-2-(4-Hydroxy-3-Methoxy- | |
| Phenyl)acetate | |
| Drinking water | Ad 100% |

### Application Example 12: Dietary Foodstuff for Special Medical Purposes (FSMP)

Production of an UHT-drink according to the recipe below.

| Ingredient | Amount in wt.-% |
|---|---|
| Whey protein concentrate (min. | 12% |
| 80% protein TS) | |
| Drinking water | Ad 100% |
| Glucose syrup | 10% |
| Vegetable oils | 4% |
| Sucrose | 2% |
| Maltodextrin | 1.5% |
| Vitamin mixture | 0.05% |
| Mineral salt mixture | 2% |
| Vanilla aroma (Symrise) | 0.2% |
| Lecithin | 0.1% |
| Citric acid | 0.05% |
| 10% 2-Ethylbutyl-2-(4-hydroxy-3- | 0.1% |
| methoxy-phenyl)acetate in ethanol | |

1-11. (canceled)

12. A compound of formula (I): 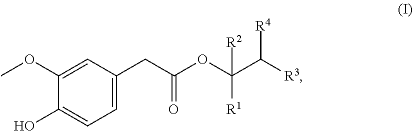
wherein
(i) R¹ and R² independently represent a hydrogen atom or an alkyl group with 1-2 carbon atoms,
R³ and R⁴ independently represent a hydrogen atom or a linear or branched alkyl group with 1 to 5 carbon atoms, a phenyl group, an alkyl phenyl group, a phenyl alkyl group, a linear or branched alkenyl group with 2 to 4 carbon atoms, an alkenyl phenyl group, or a phenyl alkenyl group,
or
(ii) R¹ and R³ form, together with the carbon atoms connecting these, a cyclohexyl ring, which is optionally substituted with a substituent R⁵, wherein R⁵ is an alkyl group with 1-2 carbon atoms,
R² represents a hydrogen atom or an alkyl group with 1-2 carbon atoms,
R⁴ represents a hydrogen atom or a linear or branched alkyl group with 1 to 5 carbon atoms, a phenyl group, an alkyl phenyl group, a phenyl alkyl group, a linear or branched alkenyl group with 2 to 4 carbon atoms, an alkenyl phenyl group, or a phenyl alkenyl group,
or a physiologically acceptable salt thereof,
wherein the phenolic hydroxyl group in formula (I) is deprotonated.

13. A compound of claim 12, wherein
R¹ and R² are each a hydrogen atom,
R³ represents a hydrogen atom, a linear or branched alkyl group with 1 to 4 carbon atoms, a phenyl group, an alkyl phenyl group, a phenyl alkyl group, an alkenyl phenyl group, or a phenyl alkenyl group, and
R⁴ is a hydrogen atom, or a physiologically acceptable salt thereof.

14. A compound of claim 13 selected from the group consisting of:
2-Phenylethyl-2-(4-hydroxy-3-methoxy-phenyl)acetate (1) 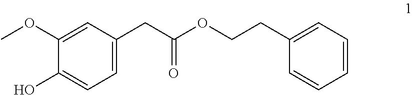 [SMILES: COc1cc(CC(=O)OCCc2ccccc2)ccc1O]
[(E)-Cinnamyl]-2-(4-hydroxy-3-methoxy-phenyl)acetate (2) 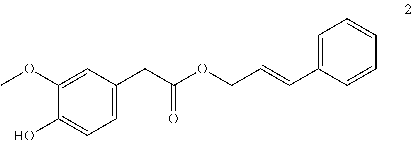 [SMILES: COc1cc(CC(=O)OC/C=C/c2ccccc2)ccc1O]
[(E)-Hex-2-enyl]-2-(4-hydroxy-3-methoxy-phenyl)acetate (5) 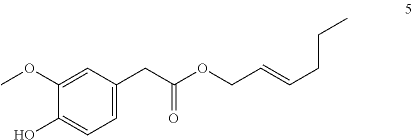 [SMILES: CCC/C=C/COC(=O)Cc1ccc(O)c(OC)c1]
[(Z)-Hex-3-enyl]-2-(4-hydroxy-3-methoxy-phenyl)acetate (6) 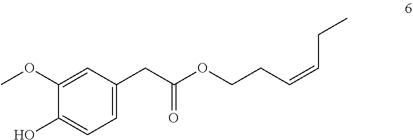 [SMILES: CC/C=C\CCOC(=O)Cc1ccc(O)c(OC)c1]
Heptyl-2-(4-hydroxy-3-methoxy-phenyl)acetate (13) 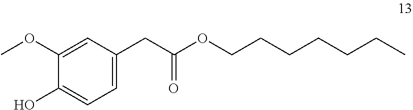 [SMILES: CCCCCCCOC(=O)Cc1ccc(O)c(OC)c1]
Ethyl-2-(4-hydroxy-3-methoxy-phenyl)acetate (16) 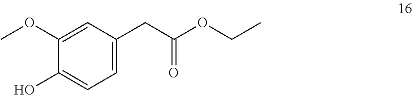
Propyl-2-(4-hydroxy-3-methoxy-phenyl)acetate (17) 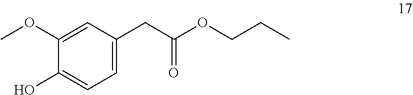 [SMILES: CCCOC(=O)Cc1ccc(O)c(OC)c1]
Butyl-2-(4-hydroxy-3-methoxy-phenyl)acetate (18) 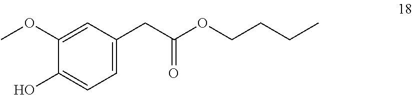 [SMILES: CCCCOC(=O)Cc1ccc(O)c(OC)c1]
Pentyl-2-(4-hydroxy-3-methoxy-phenyl)acetate (19) 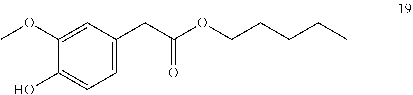 [SMILES: CCCCCOC(=O)Cc1ccc(O)c(OC)c1]
Hexyl-2-(4-hydroxy-3-methoxy-phenyl)acetate (20) 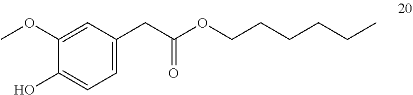 [SMILES: CCCCCCOC(=O)Cc1ccc(O)c(OC)c1]
3-Phenylpropyl-2-(4-hydroxy-3-methoxy-phenyl)acetate (21) 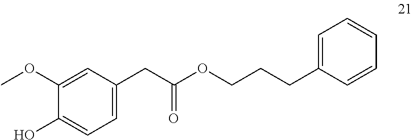 [SMILES: COc1cc(CC(=O)OCCCc2ccccc2)ccc1O]
4-Phenylbutyl-2-(4-hydroxy-3-methoxy-phenyl)acetate (22) 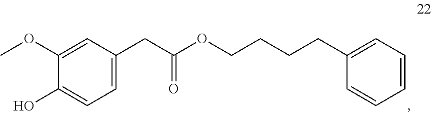 [SMILES: COc1cc(CC(=O)OCCCCc2ccccc2)ccc1O]
or a physiologically acceptable salt thereof.

15. A compound of claim 12, wherein
R¹ and R² independently represent a hydrogen atom, methyl, or ethyl,
R³ and R⁴ independently represent a hydrogen atom or a linear or branched alkyl group with 1 to 5 carbon atoms, or a physiologically acceptable salt thereof.

16. A compound of claim 12 selected from the group consisting of:
1-Ethylbutyl-2-(4-hydroxy-3-methoxy-phenyl)acetate (3) 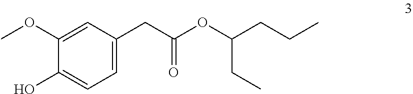 [SMILES: CCCC(CC)OC(=O)Cc1ccc(O)c(OC)c1]
3-Methylbut-2-enyl-2-(4-hydroxy-3-methoxy-phenyl)acetate (4) 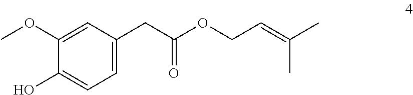 [SMILES: COc1cc(CC(=O)OCC=C(C)C)ccc1O]
Isopropyl-2-(4-hydroxy-3-methoxy-phenyl)acetate (7) 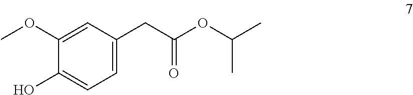 [SMILES: COc1cc(CC(=O)OC(C)C)ccc1O]
sec-Butyl-2-(4-hydroxy-3-methoxy-phenyl)acetate (8) 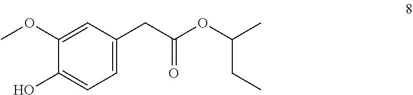 [SMILES: CCC(C)OC(=O)Cc1ccc(O)c(OC)c1]
Isobutyl-2-(4-hydroxy-3-methoxy-phenyl)acetate (9) 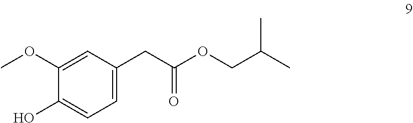 [SMILES: COc1cc(CC(=O)OCC(C)C)ccc1O]
Isopentyl-2-(4-hydroxy-3-methoxy-phenyl)acetate (10) 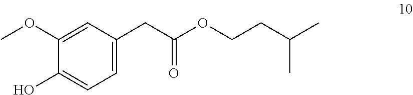 [SMILES: COc1cc(CC(=O)OCCC(C)C)ccc1O]
2-Methylbutyl-2-(4-hydroxy-3-methoxy-phenyl)acetate (11) 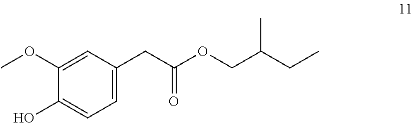 [SMILES: CCC(C)COC(=O)Cc1ccc(O)c(OC)c1]
1-Methylpentyl-2-(4-hydroxy-3-methoxy-phenyl)acetate (12) 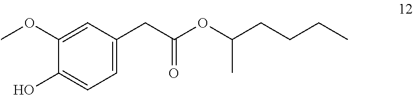 [SMILES: CCCCC(C)OC(=O)Cc1ccc(O)c(OC)c1]
1-Methylhexyl-2-(4-hydroxy-3-methoxy-phenyl)acetate (14) 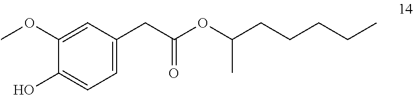 [SMILES: CCCCCC(C)OC(=O)Cc1ccc(O)c(OC)c1]
(2-Isopropyl-5-methyl-cyclohexyl)-2-(4-hydroxy-3-methoxy-phenyl)acetate (15) 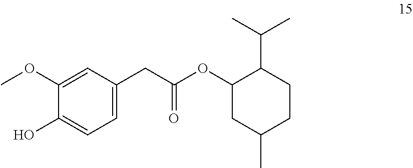 [SMILES: COc1cc(CC(=O)OC2CC(C)CCC2C(C)C)ccc1O]
2-Ethylbutyl-2-(4-hydroxy-3-methoxy-phenyl)acetate (23) 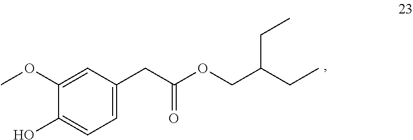 [SMILES: CCC(CC)COC(=O)Cc1ccc(O)c(OC)c1]
or a physiologically acceptable salt thereof.

17. A compound of claim 12 represented by formula (Ia) 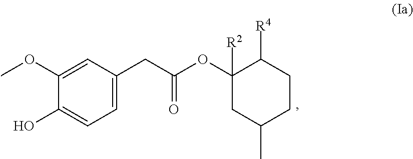
wherein,
R² is a hydrogen atom, and
R⁴ is 2-propyl, or a physiologically acceptable salt thereof.

18. A mixture comprising one or more compounds of claim 12 and/or a physiologically acceptable salt thereof.

19. An aroma composition comprising a compound of claim 12, wherein the compound is 2-ethylbutyl-2-(4-hydroxy-3-methoxy-phenyl)acetate (23) 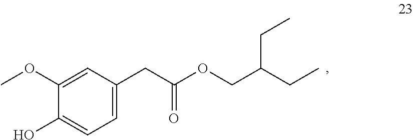 [SMILES: CCC(CC)COC(=O)Cc1ccc(O)c(OC)c1]
or a physiologically salt thereof.

20. A pharmaceutical composition comprising a compound of claim 12, wherein the compound is 2-ethylbutyl-2-(4-hydroxy-3-methoxy-phenyl)acetate (23) 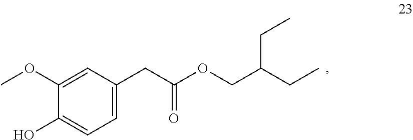 [SMILES: CCC(CC)COC(=O)Cc1ccc(O)c(OC)c1]
or a physiologically salt thereof.

21. The composition of claim 20, further comprising:
up to 99.9999 wt. %, relative to the total weight of the composition, of basic materials, excipient, and/or additives; and/or
up to 99.9999 wt. %, relative to the total weight of the composition, of water.

22. The composition of claim 20, wherein the total amount of 2-ethylbutyl-2-(4-hydroxy-3-methoxy-phenyl)acetate and/or physiologically acceptable salt(s) thereof is sufficient to inhibit or reduce the absorption of free fatty acids in the small intestine.

23. A method for producing the pharmaceutical composition of claim 20 comprising:
i) providing 2-ethylbutyl-2-(4-hydroxy-3-methoxy-phenyl)acetate (23) and/or physiologically acceptable salt(s) thereof,
ii) providing one or more further components of the pharmaceutical composition, and
iii) mixing the one or more further component of ii) with the 2-ethylbutyl-2-(4-hydroxy-3-methoxy-phenyl)acetate (23) and/or physiologically acceptable salt(s) thereof of i)).

24. A method for inhibiting or reducing the absorption of free fatty acids in the small intestine comprising administering a compound of claim 12 and/or a physiologically acceptable salt thereof to a human in need thereof.

25. The method of claim 24, wherein the free fatty acids are selected from the group consisting of saturated fatty acids with a chain length of more than 4 carbon atoms, preferably of the group consisting of butanoic acid, pentanoic acid, hexanoic acid, heptanoic aid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneiconanoic acid, docosanoic acid, tetracosanoic acid, hexacosanoic acid, octacosanoic acid, triacontanoic acid, dotriacontanoic acid, and tetratriacontanoic acid.

26. The method of claim 24, wherein the compound is 2-ethylbutyl-2-(4-hydroxy-3-methoxy-phenyl)acetate (23) 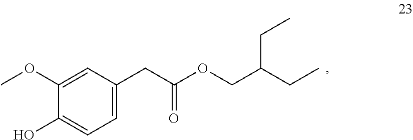 [SMILES: CCC(CC)COC(=O)Cc1ccc(O)c(OC)c1]
or a physiologically acceptable thereof.

27. A method for inhibiting or reducing the absorption of free fatty acids in the small intestine comprising administering a pharmaceutical composition of claim 20 to a human in need thereof.

28. The method of claim 27, wherein the free fatty acids are selected from the group consisting of saturated fatty acids with a chain length of more than 4 carbon atoms, preferably of the group consisting of butanoic acid, pentanoic acid, hexanoic acid, heptanoic aid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneiconanoic acid, docosanoic acid, tetracosanoic acid, hexacosanoic acid, octacosanoic acid, triacontanoic acid, dotriacontanoic acid, and tetratriacontanoic acid.

29. The method of claim 24, wherein the compound is 2-ethylbutyl-2-(4-hydroxy-3-methoxy-phenyl)acetate (23) 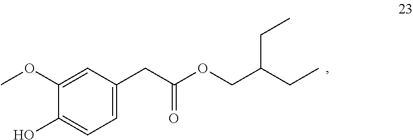 [SMILES: CCC(CC)COC(=O)Cc1ccc(O)c(OC)c1]
or a physiologically acceptable thereof.
